(19) United States
(12) Patent Application Publication
(10) Pub. No.: US 2022/0183890 A1
(43) Pub. Date: Jun. 16, 2022

(54) Solar-Powered Air Delivery System for Welder's Mask

(71) Applicant: Dane Smit, Fort Saint John (CA)

(72) Inventor: Dane Smit, Fort Saint John (CA)

(21) Appl. No.: 17/487,588

(22) Filed: Sep. 28, 2021

Related U.S. Application Data
(60) Provisional application No. 63/125,083, filed on Dec. 14, 2020.

(51) Int. Cl. A61F 9/06

(52) U.S. Cl. CPC A61F 9/068

(57) ABSTRACT

A solar powered air delivery system for a welder's mask comprises a welder's mask having a pneumatic tube terminating at an open first end within the welder's mask and terminating at a second open end about a belt. A pair of air filters and battery operated blower motor are secured to the belt and pulls air in a first direction into the second open end and out the first open end. The battery is charged by means of a solar cell disposed upon a face of the welder's mask being in electrical communication with the battery.

### RELATED APPLICATIONS

This application claims the benefit of U.S. Provisional Application No. 63/125,083, which was filed Dec. 14, 2020, the entire disclosures of which are incorporated herein by reference.

### FIELD OF THE INVENTION

The present invention relates generally to an air delivery system and more specifically to an air delivery system for a welder's mask that is solar powered.

### BACKGROUND OF THE INVENTION

Arc welding is a process that is used in a multitude of industries and hobbies. Due to the nature of the arc, a vast amount of UV radiation is released which poses a severe danger to one's eyesight. Recent technology has provided welding helmets in which the glass instantly darkens in response to the arc. While this feature makes work simpler, quick and safer, it also means that the welding helmet spends more time on a user's face during a work shift.

As these helmets are large, heavy, and thick, they also do a great job of retaining heat as well. In a hot climate, or warm and confined working conditions, this means that the user is subject to overheating. Such overheating can cause health and safety issues at the worst and poor-quality work at the least. Additionally, in cold climates, fogging can result. Accordingly, there exists a need for a means by which temperatures behind a welding mask helmet can be maintained at a comfortable level in an effort to improve safety and work efficiency. The development of the Solar-Powered Air Delivery System for a Welder's Mask fulfills this need.

### SUMMARY OF THE INVENTION

To achieve the above and other objectives, the present invention provides for an air delivery system that has a mask having a mask body with a head attachment configured to be placed on a head of a user, a first mask adjustment means which is disposed on the head attachment adapted to help secure the mask body to the user's head, at least one second mask adjustment means which is disposed on both sides of the mask body to provide an additional secure fit to the head of the user, an eyeglass which is disposed on a front of the mask body through which the user views a welding act and a workpiece and shield a user's eyes from an arc produced during the welding act, a photovoltaic cell which is disposed on an exterior of the mask below the eyeglass, a blower and a power supply electrically communicating with the solar panel, at least one filtration unit rendered in-line with the intake of the blower to provide a plurality of filtered ambient air delivered to the blower, a motor electrically communicating with the power supply and an impeller, a diffuser which is attached to a portion of an interior of the mask and a belt supporting the blower and the power supply which is attached the filtration unit.

The head attachment covers a front of the head of the user. The photovoltaic cell is capable of gathering illumination of the arc produced during the welding act and converting it into a stored form of energy. The power supply portion collects and stores the energy generated by the photovoltaic cell and provides electrical power to the blower portion upon demand and the blower includes an intake. The diffuser has a first end and a second end, the first end is in fluid communication with the second end of the delivery hose with a coupler and the second end of the diffuser is directed towards the interior of the mask and to the face of the head of the user. The belt includes a pair of distal ends and the belt is configured to encircle a waist of the user and the pair of distal ends of the belt are coupled via a securing means to secure the belt and the blower and the power supply to the waist of the user.

The front of the head of the user may include a user's face. The first mask and the at least one second mask adjustment means may be manipulated so the mask is securely fit on the head of the user. The first mask and the at least one second mask adjustment means may be manipulated so the mask is securely fit on the head of the user. The blower may be electrically communicating with the solar panel via a plurality of wiring. The blower may be a single unit storing a plurality of separate dedicated units.

The blower may be a single housing storing the separate dedicated units. The blower may include an outlet which is in fluid communication with a first end of a delivery hose. The power supply may include a back-up on-board power source. The back-up on-board power source may be a battery system. The back-up on-board power source may receive power from an outside supply that includes a selective switch that enables the user to choose between a source of power delivered to the blower portion. The blower/power supply may be disposed on a middle of the belt to provide a proper balance of weight. The mask and the blower and the power supply of the belt may ensure that there is no entanglement or obstruction of the user performing the welding act.

The at least one filtration units may be each a permanent filter. The at least one filtration units may be each a removable and replaceable filter. The motor may include a plurality of discrete settings to operate the impeller. The motor may include a plurality of variable speed settings to operate the impeller. The coupler resides outside of the mask and the coupler may be attached to the mask. The air flow from the diffuser may be directed towards a middle of the mask where it exits around the perimeter to keep temperature of the interior air the same temperature as the exterior air and prevents fogging of the eyeglass or condensation on the interior or exterior of the mask and keeps the user comfortable. The air delivery system may be worn during the welding act.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a perspective view of an air delivery system 10, according to the preferred embodiment of the present invention; and,

FIG. 2 is an environmental view of the air delivery system 10 as worn by a user 10 performing a welding act 60 on a workpiece 100, according to the preferred embodiment of the present invention; and,

FIG. 3 is sectional view of the air delivery system 10 as seen along a line I-I, as shown in FIG. 2, according to the preferred embodiment of the present invention; and,

FIG. 4, is an electrical schematic diagram of the of an air delivery system 10, according to the preferred embodiment of the present invention.

### DESCRIPTIVE KEY

10 air delivery system
20 mask
21 eyeglass
22 photovoltaic cell
23 head attachment
24a first mask adjustment means
24b second mask adjustment means
25 wiring
26 diffuser
30 blower/power supply
32 filtration unit
35 delivery hose
36 coupler
40 belt
45 securing means
50 user
51 head
52 waist
60 welding act
75 arc
100 workpiece
105 sight line
110 air flow
115 interior air
120 exterior air

### DESCRIPTION OF THE INVENTION

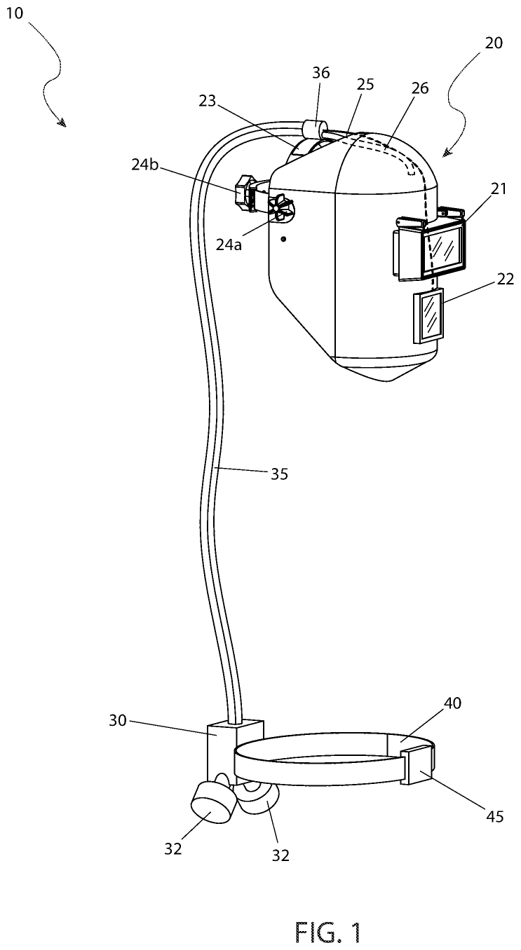

The best mode for carrying out the invention is presented in terms of its preferred embodiment, herein depicted within FIGS. 1 through 4. However, the invention is not limited to the described embodiment, and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one (1) particular configuration shall be shown and described for purposes of clarity and disclosure and not by way of limitation of scope. All of the implementations described below are exemplary implementations provided to enable persons skilled in the art to make or use the embodiments of the disclosure and are not intended to limit the scope of the disclosure, which is defined by the claims.

The terms “a” and “an” herein do not denote a limitation of quantity, but rather denote the presence of at least one (1) of the referenced items.

Referring now to FIG. 1, there is disclosed an air delivery system 10 for use with a mask 20 that would be used by a user 50 during a welding act 60. The mask 20 includes common features associated with a welder's mask, such as a mask body with a head attachment 23 configured to be placed on the head 51 of a user 50 and cover the front (i.e., the face) of the head 51. The head attachment 23 includes a first mask adjustment means 24a to help secure the mask 20 to the head 51. Similarly, on the sides of the mask 20, there may be at least one (1) second mask adjustment means 24b to provide even more secure fit to the head 51.

The front of the mask 20 includes an eyeglass 21 through which the user 50 can view the welding act 60 and workpiece 100, as well as shield their eyes from the arc 75 that is produced during the welding act 60. Below the eyeglass 21 is a photovoltaic cell 22 attached to the exterior of the mask 22. The photovoltaic cell 22 is capable of gathering the illumination of the arc 75 produced during the welding act 75 and converting it into a stored form of energy.

A blower/power supply 30 is in electrical communication with the solar panel 22 via wiring 25. The blower/power supply 30 is preferably a single unit, or a single housing storing separate dedicated units. The power supply portion collects and stores the energy generated by the photovoltaic cell 22 and provides power to the blower portion upon demand. In other embodiments, the power supply may have a back-up on-board power source, such as a battery system or can also have means to receive power from another outside supply. In these embodiments, there is a selective switch that enables the user 50 to choose between the source of power to be delivered to the blower portion.

The blower portion of the blower/power supply 30 includes an intake. Rendered in-line with the intake is at least one (1) filtration unit 32 to provide properly filtered ambient air delivered to the blower portion of the blower/power supply 30. Each filtration unit 32 can be a permanent or removable and replaceable filter. The blower portion includes a motor in electrical communication with the power supply portion and an impeller. The motor may include discrete or variable speed settings to operate the impeller. The blower portion also includes outlet which is in fluid communication with a first end of a delivery hose 35. A diffuser 25 is attached to a portion of an interior of the mask 20 and has a first end and a second end. The first end is in fluid communication with the second end of the delivery hose 35 with a coupler 36. The second end of the diffuser 25 is directed towards the interior of the mask 20 and more particularly to the face of the head 51 of the user 50. The coupler 36 may reside outside of the mask 20 or by attached thereto.

The blower/power supply 30 and attached filtration unit 32 are supported on a belt 40, which has a pair of distal ends. The belt 40 is configured to encircle a waist 52 of a user 50. The blower/power supply 30 is preferably sited in the middle of the belt 40 to provide a proper balance of weight. The distal ends of the belt 40 can be coupled via a securing means 45 so as to secure the belt 40 and blower/power supply 30 to the waist 52 of the user 50.

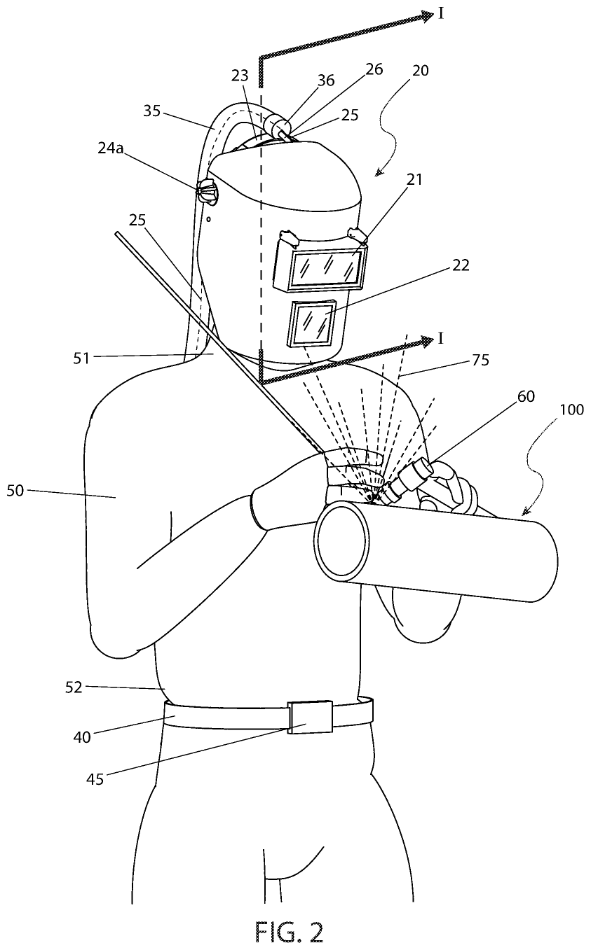

Referring now to FIG. 2, it is shown a preferred method of donning and using the sir delivery system 10. The user 20 places the head attachment 23 of the mask 20 over their head 51 such that the mask 20 fully covers the front of the head 51 (i.e., the face) and the eyeglass 22 is aligned with the eyes of the user. The first and second mask adjustment means 24a, 24b are manipulated so the mask 20 is securely fit on the head 22. The belt 40 is then placed about the waist 52 and secured thereto with the securing means 45. The user 50 then approaches the workpiece 100 and commences the welding act 60, which produces an arc 75 that the photovoltaic cell 22 on the mask 20 collects and produces energy that is directed to the blower/power supply 30 via the wiring 25. The blower/power supply 30 then is activated selectively by the generated energy from the photovoltaic cell 22, an on-board power source, or an outside power supply. The blower portion of the blower/power supply 30, once activated, provides a positive delivery of filtered air through the delivery hose 35 and to the diffuser 26, which is this directed to the interior of the mask 20 and more particularly, on the face of the head 51 of the user 50.

The positioning of the mask 20 and blower/power supply 30 of the belt 40 ensures that there is no entanglement or obstruction of the user 50 performing the welding act 60. The wiring 25 is preferably routed along or through the delivery hose 35, which is preferably two inch (2-in.) diameter tubing. The delivery hose 35 and wiring 25 runs down the back of the user 50 between the mask 20 and the blower/power supply 30.

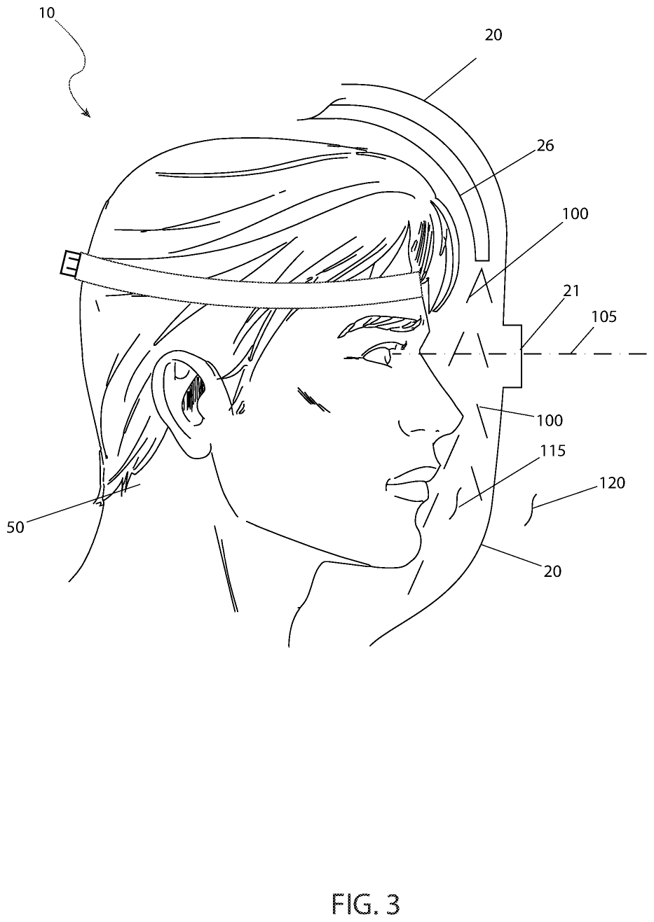

Referring next to FIG. 3, a sectional view of the air delivery system 10 as seen along a line I-I, as shown in FIG. 2, according to the preferred embodiment of the present invention is depicted. The user 50 views the welding act 60 (as shown in FIG. 2) along a sight line 105 through the eyeglass 21. Air flow 100 from the diffuser 26 is directed towards the middle of the mask 20 where it exits around the perimeter. This action keeps the temperature of the interior air 115 the same temperature as the exterior air 120 and thus prevents fogging of the eyeglass 21 or condensation on the interior or exterior of the mask 20. Additionally, the air flow 100 keeps the user 50 comfortable.

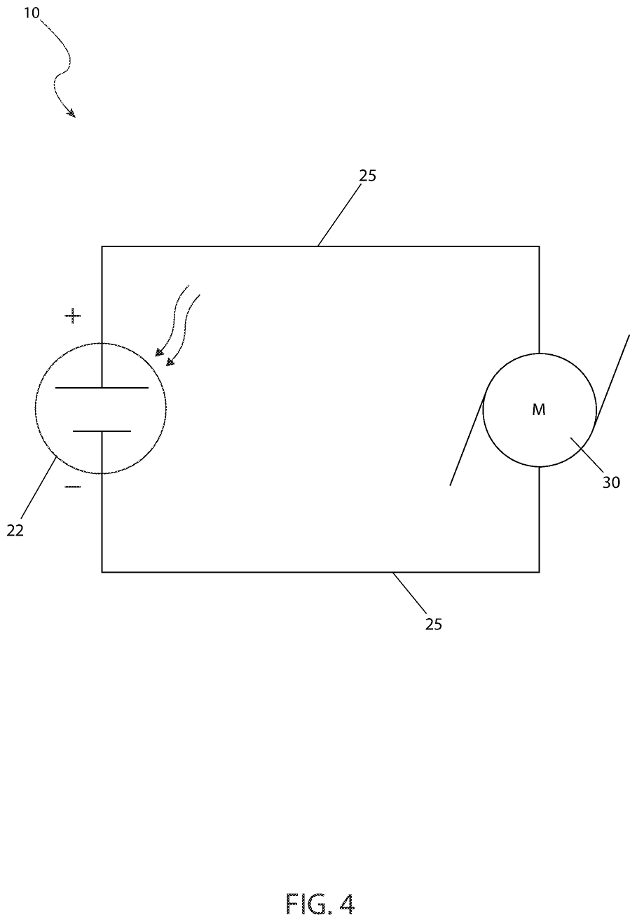

Referring finally to FIG. 4, an electrical schematic diagram of the of an air delivery system 10, according to the preferred embodiment of the present invention is disclosed. The photovoltaic cell 22 is wired to the blower/power supply 30 in a simple series circuit through the wiring 25.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

What is claimed is:

1. An air delivery system, comprising:
a mask having a mask body with a head attachment configured to be placed on a head of a user, the head attachment covers a front of the head of the user;
a first mask adjustment means disposed on the head attachment adapted to help secure the mask body to the user's head;
at least one second mask adjustment means disposed on both sides of the mask body to provide an additional secure fit to the head of the user;
an eyeglass disposed on a front of the mask body through which the user views a welding act and a workpiece and shield a user's eyes from an arc produced during the welding act;
a photovoltaic cell disposed on an exterior of the mask below the eyeglass, the photovoltaic cell is capable of gathering illumination of the arc produced during the welding act and converting it into a stored form of energy;
a blower and a power supply electrically communicating with the solar panel, the power supply portion collects and stores the energy generated by the photovoltaic cell and provides electrical power to the blower portion upon demand and the blower includes an intake;
at least one filtration unit rendered in-line with the intake of the blower to provide a plurality of filtered ambient air delivered to the blower;
a motor electrically communicating with the power supply and an impeller;
a diffuser attached to a portion of an interior of the mask, the diffuser has a first end and a second end, the first end is in fluid communication with the second end of the delivery hose with a coupler and the second end of the diffuser is directed towards the interior of the mask and to the face of the head of the user; and
a belt supporting the blower and the power supply and is attached the filtration unit, the belt includes a pair of distal ends and the belt is configured to encircle a waist of the user and the pair of distal ends of the belt are coupled via a securing means to secure the belt and the blower and the power supply to the waist of the user.

2. The air delivery system, according to claim 1, wherein the front of the head of the user includes a user's face.

3. The air delivery system, according to claim 1, wherein the first mask and the at least one second mask adjustment means are manipulated so the mask is securely fit on the head of the user.

4. The air delivery system, according to claim 1, wherein the first mask and the at least one second mask adjustment means are manipulated so the mask is securely fit on the head of the user.

5. The air delivery system, according to claim 1, wherein the blower is electrically communicating with the solar panel via a plurality of wiring.

6. The air delivery system, according to claim 1, wherein the blower is a single unit storing a plurality of separate dedicated units.

7. The air delivery system, according to claim 6, wherein the blower is a single housing storing the separate dedicated units.

8. The air delivery system, according to claim 1, wherein the blower includes an outlet which is in fluid communication with a first end of a delivery hose.

9. The air delivery system, according to claim 1, wherein the power supply includes a back-up on-board power source.

10. The air delivery system, according to claim 9, wherein the back-up on-board power source is a battery system.

11. The air delivery system, according to claim 1, wherein the back-up on-board power source receives power from an outside supply that includes a selective switch that enables the user to choose between a source of power delivered to the blower portion.

12. The air delivery system, according to claim 1, wherein the blower/power supply is disposed on a middle of the belt to provide a proper balance of weight.

13. The air delivery system, according to claim 1, wherein the mask and the blower and the power supply of the belt ensures that there is no entanglement or obstruction of the user performing the welding act.

14. The air delivery system, according to claim 1, wherein the at least one filtration units are each a permanent filter.

15. The air delivery system, according to claim 1, wherein the at least one filtration units are each a removable and replaceable filter.

16. The air delivery system, according to claim 1, wherein the motor includes a plurality of discrete settings to operate the impeller.

17. The air delivery system, according to claim 1, wherein the motor includes a plurality of variable speed settings to operate the impeller.

18. The air delivery system, according to claim 1, wherein the coupler resides outside of the mask and the coupler is attached to the mask.

19. The air delivery system, according to claim 1, wherein air flow from the diffuser is directed towards a middle of the mask where it exits around the perimeter to keep temperature of the interior air the same temperature as the exterior air and prevents fogging of the eyeglass or condensation on the interior or exterior of the mask and keeps the user comfortable.

20. The air delivery system, according to claim 1, wherein the air delivery system is worn during the welding act.
